(19) United States
(12) Patent Application Publication
(10) Pub. No.: US 2011/0008238 A1
(43) Pub. Date: Jan. 13, 2011

(54) REGENERATION OF HYDROGENATION CATALYSTS BASED ON A PLATINUM METAL

(75) Inventors: Gerard Radius, Hulst (NL); Bart Vander Straeten, St. Pauwels (BE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/920,338

(22) PCT Filed: Mar. 2, 2009
(86) PCT No.: PCT/EP09/52427

§ 371 Date: Aug. 31, 2010

(30) Foreign Application Priority Data
Mar. 3, 2008 (DE) 102008012227.0

(51) Int. Cl. C01B 21/14; B01J 38/02; B01J 38/60; C07C 51/36; C07C 45/62

(52) U.S. Cl. 423/387; 502/56; 502/27; 562/400; 568/303; 568/420

(57) ABSTRACT

A process for regenerating hydrogenation catalysts based on a platinum metal, where the hydrogenation catalysts based on a platinum metal are thermally regenerated at temperatures of from 50 to 600° C.

The present invention relates to the regeneration of hydrogenation catalysts based on a platinum metal and if necessary reduction of the amount of undesirable catalyst poisons and also to their use as hydrogenation catalysts, in particular in the preparation of hydroxylammonium salts.

For reactions proceeding in the presence of catalysts to be able to be carried out industrially, the outlay for production and regeneration, the activity, selectivity and the life of the catalyst used is of critical importance. This therefore also applies to the catalytic reduction of nitrogen monoxide by means of hydrogen in acid solution to form hydroxylammonium salts. In this process, hydrogenation catalysts comprising platinum metals are generally used.

EP-A-620 042 discloses a process for the regeneration of platinum catalysts, in which exhausted platinum metal catalysts are regenerated by bringing the platinum metal of the catalyst into solution by means of an acid or an acid mixture and, if appropriate, separating off insoluble constituents, neutralizing the platinum metal salt solution obtained and applying it to a support, if appropriate admixing it with finely divided sulfur and subsequently reducing the resulting platinum metal salt by means of a reducing agent, with, if desired, a support material being added before, during or after the treatment with finely divided sulfur. Contamination of hydrogenation catalysts based on a platinum metal with undesirable catalyst poisons generally and preferably occurs during use under reaction conditions, but can also occur during their production or regeneration. In general, the undesirable catalyst poisons are introduced into or onto the catalyst together with the starting materials, in particular with sulfuric acid. In particular, these catalyst poisons cannot be separated off from the active platinum of the catalyst or be separated off only to an unsatisfactory extent by the regeneration process according to EP-A-620 042.

It is therefore an object of the present invention to remedy the abovementioned disadvantages. A further object of the present invention may be to regenerate hydrogenation catalysts which have a specifically set, higher, lower or comparable activity and/or selectivity and/or a shorter, preferably comparable or longer operating life until the next regeneration than is/are obtained by production of the hydrogenation catalyst.

We have accordingly found a novel and improved process for regenerating hydrogenation catalysts based on a platinum metal, wherein the hydrogenation catalysts based on a platinum metal is regenerated at a temperature of from 50 to 600° C.

The process of the invention for thermally regenerating hydrogenation catalysts based on a platinum metal can be carried out as follows:

The hydrogenation catalysts based on a platinum metal can be regenerated without prepurification or preferably after prepurification at temperatures which are generally in the range from 50 to 600° C., preferably from 100 to 450° C., particularly preferably from 120 to 400° C., and preferably under an inert gas atmosphere. The duration of the thermal regeneration should generally be not less than 0.5 h and is generally from 0.5 to 10 000 h, preferably from 1 to 100 h, particularly preferably from 5 to 80 h, in particular from 12 to 60 h.

Suitable inert gases are all gases which are inert under the conditions of the thermal regeneration, e.g. nitrogen or noble gases such as helium or argon or mixtures of these gases or mixtures of gases which comprise predominantly, i.e. at least 60% by volume, preferably at least 75% by volume, particularly preferably at least 85% by volume, in particular at least 95% by volume, the inert gases. In a preferred embodiment, the proportion of oxygen in the gas phase should generally be less than 1% by volume, preferably less than 0.1% by volume, particularly preferably less than 100 ppm by volume. The pressure in the gas phase is not critical per se. Preference is given to the absolute pressure generally being in the range from 0.01 to 100 bar, preferably in the range from 0.1 to 10 bar, particularly preferably between the limits of 100 mbar below and 100 mbar above atmospheric pressure.

To prepurify the hydrogenation catalysts, the hydrogenation catalyst to be regenerated can be washed discontinuously or continuously until neutral, so that the washing liquid has a pH in the range from 5 to 8, preferably from 5.5 to 7.5, particularly preferably from 6 to 7. For this purpose, it can be advantageous to divide the total amount of washing liquid into portions and in this way to carry out washing (rinsing) in a plurality of substeps.

Suitable washing liquids are river water, if appropriate filtered river water, mains water, deionized water, in particular deionized water. The washing liquid can, if appropriate, also be partly or completely circulated, preferably partly circulated. Particular preference is given to using deionized water without circulation.

Before the thermal regeneration before or after the prepurification using a washing liquid, preferably after the thermal regeneration, the hydrogenation catalysts can be treated with a strong acid, in each case in the moist, partially dried, dried or dry state, at temperatures of from 0 to 150° C., preferably from 10 to 120° C., particularly preferably from 75 to 105° C. or at room temperature (ambient temperature) (18 to 28° C.) and an absolute pressure of from 0.1 to 100 bar, preferably from 0.5 to 50 bar, particularly preferably from 0.9 to 5 bar, in particular at atmospheric pressure, with an after-purification using a washing liquid preferably being carried out in a manner analogous to the prepurification after the treatment with strong acids.

Suitable strong acids are strong mineral acids such as nitric acid (in concentrations of from 30 to 95% by weight, preferably from 50 to 80% by weight, particularly preferably from 60 to 70% by weight, in particular concentrated nitric acid), sulfuric acid (in concentrations of from 15 to 98% by weight, preferably from 20 to 97% by weight, particularly preferably from 90 to 97% by weight, in particular concentrated sulfuric acid), hydrochloric gas (in concentrations of from 15 to 50% by weight, preferably from 20 to 45% by weight, particularly preferably from 30 to 40% by weight, in particular concentrated hydrochloric acid) or mixtures thereof, strong monocarboxylic acids such as formic acid, acetic acid or propionic acid or mixtures thereof or dicarboxylic acids such as oxalic acid or mixtures of from two to five, preferably two or three, particularly preferably two, strong acids from identical or different groups selected from among the groups mineral acids, monocarboxylic acids and dicarboxylic acids, preferably nitric acid (in concentrations of from 30 to 95% by weight, preferably from 50 to 80% by weight, particularly preferably from 60 to 70% by weight, in particular concentrated nitric acid), sulfuric acid (in concentrations of from 15 to 98% by weight, preferably from 20 to 97% by weight, particularly preferably from 90 to 97% by weight, in particular concentrated sulfuric acid), hydrochloric acid (in concentrations of from 15 to 50% by weight, preferably from 20 to 45% by weight, particularly preferably from 30 to 40% by weight, in particular concentrated hydrochloric acid) or mixtures thereof, particularly preferably mixtures of hydrochloric acid and nitric acid in a molar ratio of from 0.25:1 to 4:1, in particular aqua regia (hydrochloric acid to nitric acid in a molar ratio of 3:1), in particular obtainable by mixing the concentrated strong acids.

The following description of the regeneration can be employed both on a small scale, e.g. the laboratory scale, through to an industrial or large industrial scale in discontinuous or continuous processes, preferably in continuous processes, by replacement of the hydrogenation catalyst taken out for regeneration by freshly produced or regenerated hydrogenation catalyst. The capacity of suitable apparatuses or vessels for regeneration generally depends on the scale on which the hydrogenation process is operated and the amount of catalyst to be regenerated at the same time. The regeneration can in the case of industrial or large industrial processes be carried out continuously or discontinuously.

The thermal regeneration of the hydrogenation catalysts can be configured as storage of the catalysts in suitable apparatuses or vessels. Such an embodiment is particularly suitable in the case of industrial or large industrial processes.

Illustrative embodiments (the boundary conditions described above, e.g. temperature, pressure, starting materials and ratios, apply analogously here, even if indicated only by way of example below):

The hydrogenation catalyst can be removed or taken off in part from the reaction process before the regeneration. If appropriate, a plurality of portions of the catalyst which have been taken off are treated in succession.

The catalyst taken from the process can be washed with a washing liquid until neutral, as described above, on a suitable filtration apparatus such as a flat filter (e.g. pressure filter or flat-bed filter) or, for example, on a filter having a filter candle insert. Washing can be carried out continuously or discontinuously.

The catalyst can subsequently, if appropriate after further treatment steps such as regeneration with strong acids, be transferred into the apparatus suitable for the regeneration according to the invention, preferably without regeneration with strong acids.

Suitable apparatuses or vessels are, in particular, a preferably closed, particularly preferably gastight, cabinet into which the catalyst can be introduced and which can be supplied with gas via at least one inlet pipe and vented again via at least one outlet pipe. In this context, gastight means that, for example, a gauge pressure of 0.5 bar decreases by not more than 100 mbar, preferably only 30 mbar, with the valve closed in the inlet and outlet lines over a period of at least 30 minutes.

The gas introduced can be heated either before entry into the closed cabinet or else in the cabinet, so that the preferred regeneration temperature is reached in the gas phase. To achieve a better regeneration result, the catalyst is preferably distributed in thin layers in the cabinet, so that very uniform heating of the catalyst composition is made possible. The layer thickness can be less than 50 cm, preferably less than 15 cm, in particular less than 1 cm.

The gas flow should be selected so that very little, preferably no, fluidization of the catalyst particles occurs; in this context, fluidization refers to swirling up of the catalyst particles. Such a limit of the gas velocity is dependent on the particle size distribution of the catalyst solid and can either be estimated mathematically or determined experimentally. The gas velocity is preferably selected so that after the storage time has elapsed, the mass of the dry solid has decreased by not more than 10% by weight, preferably not more than 5% by weight, in particular not more than 1% by weight. The gas flow can be generated by means of suitable blower, for instance a compressor such as a hot-air blower or a water ring compressor or by reducing the pressure of a more highly pressurized gas to the desired pressure, e.g. reducing the pressure of nitrogen which is present in a ring system at a gauge pressure of 10 bar, for example at an orifice plate, to the desired pressure. The gas velocity can then, for example, also be adjusted by means of the design of the orifice plate to a maximum throughput and, for example, can be regulated via a manual valve or an electronically controlled valve.

The suitable apparatus can also have an offgas treatment system. This comprises, for example, a gas cooler which is able to cool the exiting hot gas to temperatures of less than 200° C., preferably less than 100° C. As gas coolers, it is possible to use conventional heat exchangers, for example air coolers, shell-and-tube heat exchangers or plate heat exchangers. The gas cooler can be followed downstream by a precipitation vessel which can precipitate any condensed gas constituents or entrained solid particles. Here, it is possible to use, for example, simple gas deflection vessels or cyclones. The configuration of the gas cooling and of the precipitation is not critical to the invention.

After the storage time has ended, the catalyst is cooled to ambient temperature under the protective gas atmosphere. The protective gas atmosphere is, in particular, maintained until the temperature of the solid has dropped below 40° C. To accelerate the cooling process, cold protective gas can be blown into the regeneration apparatus; once again, the gas velocity has to be kept below the limit for fluidization. As protective gas, preference is given to using the inert gas which was also used during heating.

The catalyst can, if appropriate, be subjected to a further treatment such as regeneration by means of strong acids as described in the present patent application before being reintroduced into the production process. Carrying out further treatment steps is not absolutely necessary, but preference is given to carrying out a treatment with strong acids before the catalyst is reintroduced into the process.

The thermal regeneration process of the invention serves to increase the activity and/or selectivity of the catalyst. It may also be able to increase the operating life of the catalyst between two regenerations in this way. A preceding decrease in the catalyst activity or selectivity or a shortening of the operating life of the catalyst can have been triggered, in particular, by catalyst poisons which get to and/or onto the catalyst together with the starting materials of the production process, in particular hydrogen, nitrogen monoxide and mineral acids such as sulfuric acid or hydrochloric acid, in particular sulfuric acid, or via the catalyst treatment process, for example the treatment with strong acids. Such poisons can come from the group of metals or metal salts, with such metals or such metals in the metal salts belonging, in particular, to the group consisting of iron, manganese, chromium, nickel, copper, aluminum, mercury. Sulfur, arsenic and selenium and also compounds comprising these elements are also known as catalyst poisons. Catalyst poisons are in particular copper, mercury, sulfur, arsenic and selenium, very particularly copper, mercury and selenium, very especially mercury, and compounds comprising these elements. Mixtures of compounds of these elements or of the elements themselves can also be catalyst poisons. In general, the elements have a sometimes strong activity-reducing effect, in particular above a threshold concentration of the respective element measured in the dry catalyst composition. These concentration limits are dependent on the individual elements and on the total poisoning of the catalyst, i.e. as a function of the concentrations of elements mentioned as catalyst poisons, based on the dry mass of the catalyst. In particular, concentrations of the individual elements mentioned as catalyst poisons of 1000 ppm by weight, preferably 500 ppm by weight, in particular 300 ppm by weight, should generally not be exceeded. The concentration of mercury or copper should generally not exceed, in each case independently of other elements or compounds of these elements which act as catalyst poisons, 1000 ppm by weight, preferably 500 ppm by weight, in particular 300 ppm by weight. To avoid exceeding these limits or to remedy this situation when a limit is exceeded, the regeneration process of the invention can be employed, in particular to reduce the concentration of mercury.

When the production quantity in a chemical process based on the use of the abovementioned hydrogenation catalyst based on a platinum metal is no longer sufficiently high despite possible multiple treatment of the catalyst with strong acids, the thermal regeneration process of the invention can be employed. A measure of the necessity of applying the thermal regeneration process of the invention to the catalyst is, apart from determination of the concentrations of the catalyst poisons on the catalyst composition, in particular that targeted poisoning of the catalyst with sulfur can no longer be carried out or be able to be carried out only to a reduced extent without the activity of the catalyst increasing to an excessive degree, i.e. not more than preferably 5%. This poisoning with sulfur is measured by dividing the amount of sulfur added in the regeneration process using strong acids by the mass of the catalyst which has been treated in this way, measured as dry mass. In particular, the thermal regeneration is employed when the amount of sulfur is less than 1000 ppm by weight, in particular less than 200 ppm by weight, especially less than 100 ppm by weight, based on the dry mass of the catalyst to be regenerated.

The thermal regeneration process of the invention is accordingly a method of increasing the activity level of the platinum metal-comprising catalyst which is suitable for a hydrogenation when this activity has decreased to an undesirable degree as a result of accumulation of catalyst poisons on the catalyst or the catalyst support. The selectivity and/or the operating life of the catalyst can also be positively influenced by the process of the invention.

The hydrogenation catalysts with which the invention is concerned can generally be obtained by treatment of a platinum salt and subsequent reduction of the platinum metal salt which has been treated in this way to metallic platinum metal.

Suitable hydrogenation catalysts based on a platinum metal include platinum metals in elemental form or on a support material, preferably on a support material.

Suitable support materials are silicon dioxide (including quartz), the aluminum oxides such as Al₂O₃, AlO(OH), calcium oxide, titanium dioxide, for example in the form of rutile, anatase, activated carbon or graphite, preferably activated carbon or graphite, particularly preferably graphite which comprises only small traces of or no catalyst poisons of the group defined at another point in the present patent application which may have been introduced by a pretreatment.

Possible platinum metal salts are in principle all platinum metal salts suitable for hydrogenation, e.g. salts of nickel, palladium, platinum, cobalt, rhodium, iridium and ruthenium, preferably palladium and platinum, particularly preferably platinum. The water-soluble salts of these metals, e.g. the halides, nitrides and sulfates, are particularly well suited. Examples which may be mentioned are:
platinum (IV) compounds such as hexachloroplatinic acid and its alkali metal and ammonium salts, tetrachloroplatinate or tetrachlorodihydroxyplatinic acid;
platinum (II) compounds such as tetrachloroplatinic acid and its alkali metal salts or platinum (II) chloride;
palladium(II) compounds such as hexachloropalladiumic acid and its salts or palladium(II) chloride.

In principle, it is also possible to use mixtures of essentially platinum metals or platinum metal salts with further metal salts:

Undesirable catalyst poisons are the elements or compounds of the elements selected from the group consisting of mercury, selenium, copper, preferably from the group consisting of mercury, copper, particularly preferably mercury.

The undesirable catalyst poisons are generally introduced into, at and onto the catalyst or the support together with the starting materials for the hydrogenation.

When the catalyst poisons on the support material decrease too quickly as a result of the thermal and/or chemical regeneration process, it is possible for the catalyst to become too active and/or too unselective.

To dampen the activity of the catalyst in a targeted manner if appropriate and increase the selectivity in a targeted manner if appropriate, catalyst poisons can be used for deliberate poisoning. This process will hereinafter be described as part of the treatment with strong acids.

Examples of suitable catalyst poisons which can be used in such a targeted manner, i.e. catalyst poisons by means of which the activity, selectivity and/or operating life of the catalyst can be set in a targeted manner and which can also be separated from the platinum in the washing and regeneration process using strong acids, are for example sulfur or sodium dithionite (Na₂S₂O₄).

As sulfur serving for partial poisoning, use is made according to the invention of finely divided sulfur, for example the commercial “flowers of sulfur”. Preference is given to using sulfur having a particle size of less than 500 μm, preferably less than 50 μm, particularly preferably sulfur having a particle size distribution in which 20% of the particles are smaller than 1 μm, 50% of the particles are smaller than 5 μm and 90% of the particles are smaller than 10 μm. (The particle size was determined by means of a MALVERN Mastersizer, see examples). Suitable sulfur is, for example, commercially available as wettable sulfur “Kumulus® WG” (BASF) or can be obtained by methods known per se, in particular sieving, from, for example, flowers of sulfur or finely milled sulfur.

In general, the platinum metal salt is treated with the finely divided sulfur in aqueous solution by bringing the aqueous metal salt solution into contact with the finely divided sulfur. The sulfur can also be used as colloidal sulfur solution (see Jander-Blasius, Einführung in das anorganisch-chemische Praktikum, 5th edition, 1964, p. 415). The sulfur is preferably added in the form of an aqueous suspension.

In principle, the preferred solvent water can also be replaced by other solvents or these can be added to the water. The sulfur can also be introduced as dry powder into the solution of the platinum metal salt.

Furthermore, substances which improve the solubility or dispersibility of the starting compounds can be added to the reaction mixture. Substances which are particularly suitable for this purpose are, on the bases of these observations, all customary surfactants in order to improve the solubility and wetting of the sulfur.

Suitable surfactants, which are also referred to as dispersants, are described, for example, in Ullmanns Encyklopädie der technischen Chemie, 4th edition, volume 23, Verlag Chemie, Weinheim, 1983, pp. 31-39. Examples which may be mentioned are:

polyacrylates, polyvinylsulfonates, polyvinylpyrrolidone, TAMOL® (BASF), Schaeffer salt and ligninsulfonates.

In a particularly preferred embodiment, ligninsulfonates (known from, for example, Ullmann, Encyclopädie der techn. Chemie, 4th edition, volume 16, p. 253 ff., Verlag Chemie, 1978), preferably alkali metal ligninsulfonates such as sodium ligninsulfonate and potassium ligninsulfonate, are used as surfactant since they can easily be removed with the washing water when the finished catalyst is washed and they do not cause pollution of the environment because they are readily degraded.

The surfactants are generally added to the reaction mixture before the addition of the sulfur to the platinum metal salt or advantageously are added to the aqueous sulfur suspension.

The weight ratio of surfactant to sulfur is generally selected in the range from 0.1 to 50% by weight, preferably from 1 to 15% by weight. On the basis of observations up to now, more than 50% by weight of surfactant does not give significant improvements in the solubility of sulfur, and amounts below 0.1% by weight generally do not result in any clearly discernible improvement.

The temperature during the treatment of the platinum metal salt with the finely divided sulfur is usually selected in the range from 20 to 95° C., preferably from 40 to 95° C., particularly preferably from 50 to 85° C.

The pH during the treatment of the platinum metal salt with the finely divided sulfur is generally selected in the range from 1.5 to 11.5, preferably from 2.5 to 8.5, particularly preferably 4.5 to 8.5 very particularly preferably from 5.6 to 6.2.

The platinum metal salt is neutralized by means of Na₂CO₃ to a pH of 3.0. The solution is then buffered with sodium acetate until a pH of from 5.6 to 6.2 is reached.

The duration of the treatment of the platinum metal salt with the finely divided sulfur, i.e. the period of time from the addition of the finely divided sulfur to the addition of the reducing agent, is generally selected in the range from 0.5 to 60 min, preferably from 2 to 15 min. A treatment shorter than 0.5 min generally leads to insufficient poisoning of the catalyst, while a treatment longer than 60 min gives, on the basis of previous experience, no advantages.

The mass ratio of sulfur to platinum metal is generally selected in the range from 0 to 30% by weight, preferably from 0.5 to 15% by weight.

After the partial poisoning with sulfur, the platinum metal salt is reduced to metallic platinum metal, advantageously by adding a reducing agent to the reaction mixture obtained after treatment of the platinum metal salt with finely divided sulfur.

Possible reducing agents are usually all known reducing agents for reducing platinum metal salts to platinum metal, e.g. hydrazine, formaldehyde, formic acid or an alkali metal formate or alkaline earth formate, e.g. sodium, potassium and calcium formate, particularly preferably formic acid.

The molar ratio of reducing agent to platinum metal is generally selected in a molar excess, preferably at least two-fold, preferably at least 10 fold, particularly preferably at least 40 fold molar excess.

The temperature during the reduction is generally selected in the range from 20 to 98° C., preferably from 40 to 95° C., particularly preferably from 50 to 90° C.

After the reaction is complete, the catalyst is generally worked up in a customary fashion, for example by filtering it off from the reaction mixture and advantageously washing it with water, preferably until the continuously or discontinuously discharged washing water has a pH in the range from 5.0 to 7.0, particularly preferably from 6.0 to 7.0.

The regeneration using strong acids thus comprises

a) treatment of the catalyst with strong acids,
b) if appropriate, neutralization of the regeneration solution,
c) setting of a desired degree of poisoning with a catalyst poison such as sulfur,
d) reduction of the platinum,
e) if appropriate, washing of the regenerated catalyst with water until neutral.

The catalyst is ideally but not necessarily washed with water until neutral before the regeneration using strong acids. The thermal regeneration according to the invention can then be carried out before the regeneration using strong acids is carried out.

In a preferred embodiment, the reduction and, if desired, the treatment with finely divided sulfur is carried out in the presence of a catalyst support such as graphite or activated carbon, preferably graphite. The platinum metal salt is particularly preferably admixed with finely divided graphite, generally graphite where more than 90% by weight, preferably more than 95% by weight have a particle size in the range from 0.1 to 1000 μm, preferably from 1 to 300 μm, particularly preferably from 2 to 100 μm, before the treatment with finely divided sulfur.

The molar ratio of platinum metal to carbon (or graphite or activated carbon) is generally selected in the range from 0.001 to 10% by weight, preferably from 0.01 to 5% by weight, particularly from 0.05 to 2% by weight.

The catalysts obtained by the process of the invention are, on the basis of previous observations, suitable for the hydrogenation of both organic and inorganic compounds.

The catalysts according to the invention are preferably used for the hydrogenation of olefinically or acetylenically unsaturated compounds, such as C₂-C₁₂-olefins and C₂-C₁₂-alkynes, preferably water-soluble C₂-C₁₂-olefins and C₂-C₁₂-alkynes, particularly preferably aliphatic C₂-C₁₂-olefins and aliphatic C₂-C₁₂-alkynes, very particularly preferably propene, butenes, pentenes, propyne, butines, pentines, also for the hydrogenation of carboxylic acids such as C₂-C₁₂-monocarboxylic acids and C₄-C₁₂-dicarboxylic acids, preferably water-soluble C₂-C₁₂-monocarboxylic acids and water-soluble C₄-C₁₂-dicarboxylic acids, particularly preferably acetic acid, propionic acid, butyric acid, glutaric acid, adipic acid, aldehydes such as C₂-C₁₂-aldehydes, preferably water-soluble C₂-C₁₂-aldehydes, particularly preferably ethanal, propanal, butanals, pentenals, hexenals, or ketones to the corresponding alcohols, or of nitriles to the corresponding amines. Furthermore, the catalysts according to the invention are suitable for the hydrogenation of inorganic substances such as oxygen, but in particular for the preparation of hydroxyammonium salts by hydrogenation of nitrogen monoxide in aqueous mineral acids.

The preparation of hydroxyammonium salts is generally carried out at a molar ratio of hydrogen to nitrogen monoxide of from 1.5:1 to 6:1, preferably from 1.6:1 to 5:1.

The hydrogenation of nitrogen monoxide is generally carried out at a temperature in the range from 30 to 80° C., preferably from 35 to 60° C. Furthermore, the pressure during the hydrogenation is usually selected in the range from 1 to 30 bar (absolute), preferably from 1.3 to 10 bar (absolute).

The ratio of catalyst to mineral acid depends essentially on the platinum metal and on the reactor pressure and in the case of platinum is generally in the range from 5 to 100 g, preferably from 10 to 30 g, of platinum/graphite catalyst per liter of mineral acid.

In a further preferred embodiment, especially in the preparation of hydroxylammonium salts, the catalyst is treated with hydrogen (“activation”) in acid solution, advantageously in the mineral acid in which the hydrogenation is carried out, before the hydrogenation.

The catalysts according to the invention are, on the basis of observations to date, superior in terms of activity, selectivity and operating life to known catalysts for the same purpose, insofar as the two have not been repoisoned in a targeted manner by means of, for example, sulfur. In the case of targeted poisoning, the catalysts which have been regenerated according to the invention require addition of more sulfur in order to achieve the same activity, selectivity or operating life than a catalyst which has not been regenerated according to the invention and has been taken from the same basic totality of catalyst. The process of the invention for producing and regenerating hydrogenation catalysts, additionally has the advantage that the amount of waste catalyst is reduced because of the longer time for which the catalyst can be used. Less catalyst per unit time has to be discharged and disposed of.

### EXAMPLE 1

### Determination of Particle Sizes

The particle size was determined using a MALVERN Mastersizer (see also Verfahrenstechnik 24 (1990) p. 36ff). The Fraunhofer diffraction at a wavelength of 633 nm was measured. The particle size distribution was determined in a range from 1 to 600 μm by choice of a front lens having a focal length of f=300 mm.

To carry out the measurement, a spatula tip of the powder to be examined was added to 1 liter of a 0.1% strength by weight aqueous Nekanils 910 solution (BASF AG; Nekanil® 910 is a nonylphenol reacted with 9-10 mol of ethylene oxide; properties: clear, colorless, viscous liquid; nonionic, density at 20° C.: 1.04 g/cm³; pour point: below −10° C.; pH of a 1% strength by weight solution: 6.5 to 8.5). Before the measurement, the mixture to be examined was subjected to treatment with ultrasound for 1 minute.

### EXAMPLE 2

### Thermal Regeneration

Thermal regeneration of a hydrogenating catalyst based on a platinum metal partially poisoned with finely divided sulfur.

Thermal regeneration is carried out using an amount of 920 kg of moist catalyst. The catalyst is distributed over various containers and introduced into an oven. The 920 kg are distributed over 60 containers.

When the oven has been filled, the temperature program is started.

The temperature program follows the temperatures in the table below:

| | | Final temperature of | Duration |
|---|---|---|---|
| Phase | | the phase (° C.) | (h) |
| 0 | Start | 20 | 0 |
| 1 | Heating ramp | 140 | 2 |
| 2 | Hold | 140 | 10 |
| 3 | Heating ramp | 400 | 2 |
| 4 | Hold | 400 | 48 |
| 5 | Cooling | 30 | 4 |

The thermal regeneration is monitored by means of analytical measurements before and after the treatment. The important aspect here is the improvement in the activity of the catalyst which is measured by chemisorption on platinum. Before the treatment, chemisorption values in the range from 2000 cm²/g to 5000 cm²/g (plurality of random samples from the catalyst composition) are measured, typically 2500 cm²/g. After the treatment, values of from 5000 cm²/g to 8000 cm²/g are achieved, typically 7000 cm²/g.

The content of mercury on the graphite before the thermal regeneration is in all samples from 200 to 400 mg/kg and after the regeneration according to the invention is from 10 to 50 mg/kg, typically 20 mg/kg.

The reaction rate of the catalyst was determined before the thermal regeneration. The rate was 0.01 mol of N/h/g_{cal}. After the thermal treatment, the reaction rate increases to 0.09 mol of N/h/g_{cal}.

### EXAMPLE 3

### Chemical Regeneration

The same catalyst which was used for the thermal regeneration (further 920 kg of moist composition) is chemically regenerated.

When carrying out the chemical regeneration without thermal regeneration, it is not possible to increase the specific platinum surface area nor decrease the content of mercury. The chemisorption measurements are from 2000 cm²/g to 2800 cm²/g before regeneration. After chemical regeneration, the chemisorption is unchanged at from 2000 cm²/g to 2800 cm²/g. The mercury content remains unchanged at from 200 to 400 mg/kg, typically 350 mg/kg, before and after the chemical regeneration.

### EXAMPLE 4

### Chemical Regeneration after Thermal Regeneration

After the thermal regeneration in example 3, a portion of 320 kg of the catalyst which has been treated in this way is chemically regenerated. The platinum surface areas and mercury concentrations described in example 3 remain in the range obtained in example 3 for all random samples.

1-6. (canceled)

7. A process for regenerating hydrogenation catalysts based on a platinum metal, wherein the hydrogenation catalysts based on a platinum metal are thermally regenerated at temperatures of from 50 to 600° C. under a protective gas atmosphere.

8. The process for regenerating hydrogenation catalysts based on a platinum metal according to claim 7, wherein the hydrogenation catalysts based on a platinum metal are subsequently treated with strong acids after the thermal treatment.

9. The use of the hydrogenation catalysts based on a platinum metal which have been regenerated according to claim 7 for the hydrogenation of organic or inorganic compounds.

10. The use of the hydrogenation catalysts based on a platinum metal which have been regenerated according to claim 9, wherein nitrogen monoxide as inorganic compound is hydrogenated to hydroxylammonium salt.

11. The use of the hydrogenation catalysts based on a platinum metal which have been regenerated according to claim 9, wherein olefinically or acetylenically unsaturated compounds or carboxylic acids, aldehydes or ketones as organic compounds are hydrogenated.

12. The use of the hydrogenation catalysts based on a platinum metal which have been regenerated according to claim 8 for the hydrogenation of organic or inorganic compounds.

13. The use of the hydrogenation catalysts based on a platinum metal which have been regenerated according to claim 12, wherein nitrogen monoxide as inorganic compound is hydrogenated to hydroxylammonium salt.

14. The use of the hydrogenation catalysts based on a platinum metal which have been regenerated according to claim 12, wherein olefinically or acetylenically unsaturated compounds or carboxylic acids, aldehydes or ketones as organic compounds are hydrogenated.
